(19) United States
(12) Patent Application Publication
(10) Pub. No.: US 2021/0249110 A1
(43) Pub. Date: Aug. 12, 2021

(54) ENCOUNTER-BASED ELECTRONIC MEDICAL RECORD

(71) Applicant: International Business Machines Corporation, Armonk, NY (US)

(72) Inventors: Abhishek Malvankar, White Plains, NY (US); Parthasarathy Suryanarayanan, Croton on Hudson, NY (US); Edward A. Epstein, Putnam Valley, NY (US); Burn Lewis, Ossining, NY (US)

(21) Appl. No.: 16/785,873

(22) Filed: Feb. 10, 2020

(51) Int. Cl. G16H 15/00; G16H 10/60; G06Q 10/10; G06F 16/23

(52) U.S. Cl. CPC G16H 15/00; G06F 16/2379; G06Q 10/10; G16H 10/60

(57) ABSTRACT

A method of managing an electronic medical record comprises receiving patient data associated with the medical record. First encounter information in the patient data is identified and compared with second encounter information in a first encounter bundle in the medical record. Based on the comparing, the first encounter information and second encounter information are determined to be associated with the same encounter. The patient data is added to the first encounter bundle based on the determining. The first encounter bundle is analyzed, resulting in a computer-readable artifact for the first encounter bundle. A summary of the first encounter bundle is updated based in part on the computer-readable artifact.

### BACKGROUND

The present disclosure relates to electronic medical records, and more specifically, to summarizing medical records in real time.

Medical records, including electronic medical records, can contain a significantly large amount of information. Doctors and other medical professionals often are required to review a patient's medical records at least once, and sometimes frequently, while treating a patient. Due to the significant amount of information in some medical records, reviewing all medical information in a medical record can be challenging. Further, because some medical records are updated frequently, doctors may need to constantly review a medical record to keep up-to-date knowledge of a patient's medical history.

### SUMMARY

Some embodiments of the present disclosure can be illustrated as a method that comprises receiving, by a computer system that manages an electronic medical record, patient data associated with the medical record. The method also comprises identifying first encounter information in the new patient data and comparing the first encounter information with second encounter information in a first encounter bundle in the medical record. The method also comprises determining, based on the comparing, that the first encounter information and the second encounter information are associated with the same encounter. The method also comprises adding, based on the determining, the patient data to the first encounter bundle. The method finally comprises updating, based in part on the computer-readable artifact, a summary of the first encounter bundle.

Some embodiments of the present disclosure can also be illustrated as a system comprising a processor and a memory in communication with the processor, the memory containing program instructions that, when executed by the processor, are configured to cause the processor to perform a method. The method may comprise receiving first patient data associated with a medical record. The method may also comprise identifying first encounter information in the first patient data and comparing the first encounter information with second encounter information in a first encounter bundle in the medical record. The method may also comprise determining, based on the comparing, that the first encounter information and the second encounter information are not associated with the same encounter. The method may also comprise creating, based on the determining, a second encounter bundle in the medical record. The method may also comprise adding, based on the determining, the first patient data to the second encounter bundle. The method may also comprise analyzing the second encounter bundle, resulting in a first computer-readable artifact that for the second encounter bundle. Finally, the method may include creating, based in part on the first computer-readable artifact, a summary of the second encounter bundle.

Some embodiments of the present disclosure can be illustrated by a computer program product, the computer program product comprising a computer readable storage medium having program instructions embodied therewith, the program instructions executable by a computer to cause the computer to perform the method above that is performed by the above system.

The above summary is not intended to describe each illustrated embodiment or every implementation of the present disclosure.

### BRIEF DESCRIPTION OF THE DRAWINGS

The drawings included in the present application are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 illustrates an example method of a target update to a summary of an encounter bundle, in accordance with embodiments of the present disclosure.

FIG. 2 illustrates a server that may store the data of a patient's electronic medical record organized into encounter bundles, in accordance with embodiments of the present disclosure.

FIG. 3 illustrates an example flow diagram in which two pieces of data are processed upon being added to a medical record, in accordance with embodiments of the present disclosure.

FIG. 4 depicts the representative major components of a computer system that may be used in accordance with embodiments of the present disclosure.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

Aspects of the present disclosure relate to electronic medical records, and more specifically, to summarizing medical records in real time. While the present disclosure is not necessarily limited to such applications, various aspects of the disclosure may be appreciated through a discussion of various examples using this context.

Providing medical treatment for a patient, in many medical fields, involves a review of that patient's medical record before the patient is treated. In some instances this review may help to identify the proper treatment of a patient or to avoid mistreating a patient. For example, some medical advice, medications, surgeries, or other treatments may be counterindicated by information in that patient's record. In other instances, historical information may be present in a patient's medical record that may be combined with new information to help to identify an affliction that may have been undiagnosed for years of a patient's life.

For example, if a patient who is suffering from a long-term affliction is seeing a new medical professional for that affliction, that medical professional may prescribe a common medication for that affliction. However, if a previous professional had prescribed that same medication to the patient and it did not improve the affliction (or resulted in significant negative side effects), prescribing that medication may be considered medically irresponsible, even malpractice. On the other hand, upon reviewing the patient's medical history, a medical professional may recognize previous test results that were inconclusive at the time they were produced, but that due to changed circumstances (e.g., new test results, new medical studies), the previous test results may imply to the medical professional that the patient is suffering from a rare condition that can be treated with other methods. Thus, by reviewing the patient's medical record, the medical professional may have not only avoided a potential medical malpractice claim, but also identified an effective treatment for the long-term affliction that had, up until that point, remain untreated.

Unfortunately, a typical patient's medical record can be very long and contain information from various providers, much of which may be largely irrelevant to any particular medical professional's relationship with the patient. Further, some of the information in the typical health record may be inaccurate, duplicative, or out of date. Even assuming, hypothetically, that all the data in a medical record were accurate and up to date, the data that is relevant to any particular medical provider may oftentimes be buried in unstructured data (e.g., clinical notes), and may be difficult to find quickly. Thus, the medical records of even the typical patient may be very difficult to review in a reasonable amount of time.

Further, many patients have medical records that are far longer, far more varied, and far more frequently updated than the typical patent's record. Performing a review of these medical records that is thorough enough for a medical professional to identify the relevant or helpful information in the medical record may be impossible in the amount of time that a medical professional may have available to dedicate to such a review. Thus, in some instances the information in medical records that is necessary to avoid potential malpractice claims or to effectively treat a patient may not be taken advantage of, or may be taken advantage of too late.

For these reasons, it can be beneficial to summarize a patient's medical record before providing that medical record to a medical professional for review. Such a summary may enable a medical professional to review the important aspects of a patient's medical record quickly, significantly increasing the likelihood that the information therein may improve the treatment given to the patient. For example, an electronic version of a patient's medical record may be analyzed by a computer (e.g., one or more processors, a generic computer, or a neural network) and summarized in various ways. For example, a medical record may be separated into sections (e.g., lab results, current medication prescribed, historical medications prescribed, diagnoses, vital measurements, previous surgeries, etc.) which may then be presented to a medical professional or summarized and presented.

Unfortunately, some medical records are constantly updated, causing the summaries thereof to become out of date regularly. For example some medical conditions require periodic treatment (e.g., weekly chemotherapy, radiation or a mixture of both), and thus the medical records of patients undergoing such medical treatments may become outdated on the same periodic schedule. In another example, some medical conditions may require a patient to regularly visit a large medical facility for several days at a time, during which time that patient may see multiple medical professionals, have multiple lab tests run, and undergo multiple procedures every day. In some examples a patient's medical record may be updated every day or multiple times every day. In these examples, a summary of a patient's medical record may require very frequent updating in order to be most helpful to a reviewing medical professional.

However, updating a summary of a medical record sometimes requires significant processing resources. Some medical records may contain years' worth of structured data (e.g., computer-readable forms) and unstructured data (e.g., hand-written notes, medical images such as x-rays and MRI images, natural-language text, voice memos, and others). In some instances, the updating process may require several artificial-intelligence analysis systems (e.g., machine-learning systems, neural networks, rule-based analysis systems) to digest several types of unstructured data and convert that unstructured data into computer-readable information. This process of digestion and conversion may sometimes utilize information available in structured data to expedite the digestion process or make it more feasible. For example, the meaning of a note in unstructured data may not be clear unless the image recognized within that note is analyzed in light of a piece of structured data from the record (e.g., a piece of structured data describing the patient as anemic or categorizing the note as written by the patient's surgeon).

The converted unstructured data may be referred to herein as “artifacts” or as “machine-readable artifacts” produced by the analysis. These artifacts, together with the medical-record structured data, may then be reviewed by a computer system to create a summary of the medical record. In some use cases, these artifacts are discarded after the summarization process, and new artifacts may be reproduced when the medical record is updated and an updated summary is necessary.

However, it may take a significant amount of time for a artificial-intelligence analysis system (sometimes referred to herein as an AI analysis system) to analyze a medical record with a large amount of unstructured data and produce artifacts for summarization. In some instances, a medical record may be large enough, updated frequently enough, or both, that frequent updates to a summary of the medical record is unfeasible. In some instances, for example, it may be financially infeasible to provide the hardware necessary. In other instances, the hardware may be available, but keeping up with the updates to the medical record may require continuous high performance of that hardware, which may increase operational costs to an undesirable degree. For example, there may be large financial costs associated with powering the computer system, financial costs of replacing equipment that fails due to the high demands placed upon it, and operational costs of being unable to use the computer system for other purposes. In some instances, it may simply not be possible for a modern computer system to analyze a medical record (or set of medical records) and produce summaries thereof quickly enough. This may negatively impact the ability to provide real time (or even near-real time) summaries of these medical records. This may also be exacerbated in use cases in which artifacts are discarded after summaries are created, because any analysis of unstructured items that is necessary to prepare the summaries would need to be performed each time a summary is updated.

Thus, there is a need to address the challenges associated with updating summaries of a medical record in near-real-time. One potential method of addressing these challenges includes storing the artifacts produced by analysis of unstructured data when those unstructured data are analyzed. For example, after a medical record is analyzed, the computer-readable artifacts are created, and a summary is created, the artifacts could be stored in a file that is appended to the medical record. This file may then be accessed when updating the summary of the medical record. Thus, because the computer-readable artifacts of the unstructured data would already be available, repeating the analysis of much of the unstructured data in the medical record would be unnecessary. By skipping this step, the process of updating a summary of a medical record may take significantly less time, which may increase the ability to provide near-real-time updates to summaries of medical records.

However, in some instances there may be negative consequences associated with storing computer-readable artifacts produced by analyzing the unstructured data of a medical record. For example, as previously discussed, some medical records may have significantly large amounts of unstructured data gathered after, for example, years of regular medical appointments and procedures. The analysis of these unstructured data may, as a result, create a large amount of artifacts to be stored. Storing these artifacts, then, may require a large amount of storage space. The cost of acquiring and maintaining this storage space, while it may enable faster updates of summaries medical records, may be financially undesirable or even infeasible. This may be exacerbated for storage systems that are responsible for maintaining the medical records (or summaries thereof) for multiple individuals. This may, therefore, make operating a system that maintains the electronic medical records of many users challenging.

Thus, storing artifacts produced by analysis of unstructured data may not sufficiently address the challenges associated with updating summaries of a medical record in real time. One further method of addressing these challenges includes organizing the data within the electronic medical record in such a way that enables targeted analysis of the data within the medical record when summaries need to be updated. In typical electronic medical records, most, if not all, data may be grouped together, allowing all the data to be analyzed together and compared. When data is added to the electronic medical record, due, for example, to a new appointment (e.g., a doctor's appointment) or to an update resulting from a past visit (e.g., a set of test results returned a manner of weeks after a hospital stay), all data in the medical record may be reanalyzed to create an updated summary. This reanalysis may include, as previously discussed, recreating computer-readable artifacts from unstructured data or simply reanalyzing the previously stored artifacts of previous analyses. In either scenario, the reanalysis may spend significant portions of time reanalyzing portions of the medical record that are unaffected by the data that is added to the medical record.

Some embodiments of the present disclosure organize the data in an electronic medical record based on the medical encounter from which that data was created. An encounter, as used herein, may refer to one or more experiences with a medical professional, such as a doctor's visit, a meeting with multiple doctors, a hospital stay, and others. An encounter bundle (for example a directory or a file that contains data relevant to that encounter) may be created in the medical record for each encounter in the record, and data within the medical record may be organized into encounter bundles based on the encounter that resulted in the data being created.

The specificity with which encounters are defined may vary between medical records or between systems that maintain medical records. For example, a medical record may contain data related to a patent's hospital stay that spanned three days. The overall stay may have included a surgery, lab testing, and several visits with doctors, some of which may have been with a single doctor and some of which may have been with multiple doctors simultaneously (e.g., a medical team). In some embodiments, for example, a single encounter bundle may be created for this hospital stay, and all data resulting from the surgery, doctor visits, and lab results may be added to the single encounter bundle. In other embodiments, three separate encounter bundles may be created: one for each day of the hospital stay. In other embodiments, one encounter bundle may be created for the surgery, and one for each doctor's visit. In some embodiments, one encounter bundle may be created for a visit with multiple doctors, whereas in other embodiments a separate encounter bundle may be created for each doctor present during the visit. In some embodiments, test results (e.g., the lab results) may be placed into the encounter bundle that also contains the data pertaining to the doctor's visit that ordered the test be performed. In other embodiments, an encounter may be created for the test that created the results. This may be useful, for example, in instances in which multiple tests are performed on a single visit to a testing center.

Regardless of the specificity with which encounters are defined, the data of a medical record that are relevant to a particular encounter may be stored in an encounter bundle for that encounter. For example, medical data received for the electronic medical record may include structured data from which information identifying the relevant encounter may be obtained. For example, a medical form on which notes are typed or written may include a field that specifies the encounter (e.g., the doctor's visit) to which the notes are related, and an X-ray image may include metadata that specifies the same.

A summary of the encounter bundle may then be created by analyzing the structured and unstructured data within that encounter bundle. When data is added to the medical record, the computer system may identify encounter information in the new data and determine whether a matching encounter bundle exists in the medical record. If a matching encounter bundle is found, the new data may be added to the matching encounter bundle. If no matching encounter bundle is found, a new encounter bundle may be added to the medical record, and the new data may be added to that new encounter bundle.

Once new data is added to the relevant encounter bundle, a summary for that encounter bundle may, in some embodiments, be created immediately and stored for later retrieval. In some embodiments, for example, a database may store a separate summary for the most-updated version of each encounter bundle. In other embodiments, a summary for the encounter bundle may not be created until it is requested by a medical practitioner.

For example, when the summary of the medical record is updated, only the encounter bundle to which the new data has been added may be reanalyzed. If the new data had been added to an existing encounter bundle, the structured and unstructured data (including the new data) of that encounter bundle may be reanalyzed, but no analysis of other encounter bundles may be performed. A summary of that encounter bundle may then be updated using the results of the reanalysis of the updated encounter bundle. Because only a partial amount of the medical record is reanalyzed, it may be possible to perform this targeted summary update very quickly without requiring the expensive storage of previous analysis artifacts. Particularly in large medical records with a large amount of encounter bundles, the time savings may be significant, and may enable the real-time (or near-real-time) updates of medical-record summaries.

Thus, this encounter bundle organization may enable a targeted update to one or more summaries of the medical record (e.g., only the summaries of encounter bundles that have themselves been updated), which may significantly reduce the time and resources necessary for maintaining a summary (or summaries) of the entire medical record, as well as reduce or eliminate the need to store the artifacts of previous analyses.

Further, the capability to efficiently maintain a separate summary for each encounter bundle may improve the capability to respond to real-time requests of medical professionals to view a summary of selected portions of the medical record (e.g., a summary of data solely pertaining to a single encounter or to a set of encounters). For example, rather than request a summary of the entire medical record, a medical practitioner may request a summary of the most recent encounter, all encounters over the past month, all encounters at that medical practitioner's office, or all encounters related to a leg injury. In any of these examples, a computer system that manages the medical record may identify the corresponding encounter bundles, obtain the summaries of those bundles, and deliver them to the medical practitioner. In some embodiments, the summaries may be delivered as distinct items (e.g., separate folders or compressed files) or may be merged, so the medical practitioner receives a single summary to view. In some embodiments, the requested summaries may be readily available to the computer system, whereas in other embodiments one or more of the summaries may need to be updated due to new data being stored in the corresponding encounter bundle.

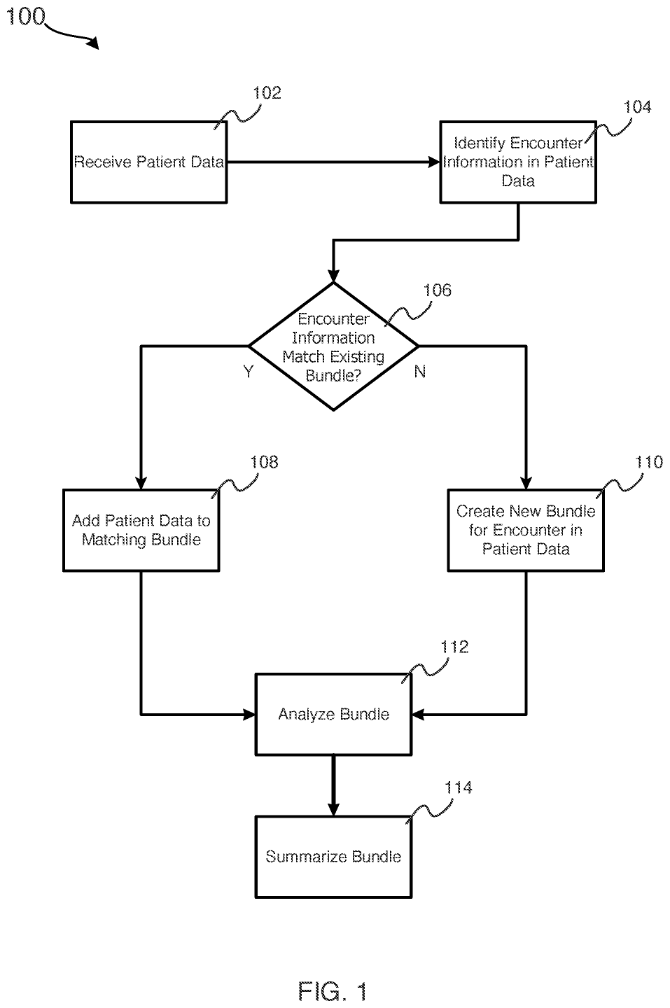

FIG. 1 illustrates an example method 100 of a target update to a summary of a medical record that is organized into encounter bundles. Method 100 may be performed by a computer system (or a set of computer systems) that may include, for example, a server, computer processors, graphics processors, and even dedicated circuitry designed to operate machine-learning algorithms such as neural networks. In block 102, the computer system receives patient data for the medical record. This patient data may be, for example, notes taken by a doctor following a doctor's visit, correction to previous data in the medical record, results of one or more tests run on a patient, or others.

In block 104, the computer system may identify encounter information (sometimes referred to herein as the “identified encounter information”) in the patient data. This encounter information may include, for example, a date or time of a doctor's visit, a date of a hospital stay, a location of the encounter (e.g., a specialist's office name or address) information identifying the health provider, or other information from which the encounter can be derived.

In block 106, the computer system determines whether the identified encounter information matches any of the encounter bundles in the medical record. This may include, for example, scanning the encounter bundles to determine a best match between the encounter information in each bundle and the identified encounter information. This may also include, for example, comparing the identified encounter information to an encounter-bundle label (e.g., a name of the encounter bundle or a meta-data tag attached to the encounter bundle) of each encounter bundle. This label may describe the encounter associated with that encounter bundle with sufficient specificity that a match may quickly be confirmed or rebutted.

If a match is identified in block 106, the computer system adds the patient data to the matching bundle in block 108. In some embodiments, this may also include updating a version number of the bundle, which may be useful for tracking the development of the medical record over time.

If a match is not identified in block 106, the computer system creates, in block 110, a new encounter bundle for the encounter that is associated with the identified encounter data (and that therefore pertains to the patient data). The computer system then adds the patient data to the newly created encounter bundle.

Once the patient data is added to an encounter bundle (either in block 108 or block 110), the computer system can analyze, in block 112, the data in the encounter bundle to which the patient data has been added. This analysis may include, for example, creating computer-readable artifacts from the unstructured data within the encounter bundle. For example, the encounter bundle may include handwritten notes, typed notes, test results, voice memos, and MRI images. In this example, the analysis in block 112 may include image recognition to identify letters, numbers, and words in the handwritten notes, then performing natural-language processing on those identified letters, numbers, and words. The analysis may also include performing natural language analysis on the typed notes and test results. The analysis may also include performing audio analysis on the voice memos and natural language analysis on any identified words and numbers. The analysis may also include performing image analysis on the MRI images to determine whether any known conditions are identified therein. These analyses, for example, may be performed by one or more neural networks that have been trained to recognize patterns in the unstructured data.

Analysis of the encounter bundle in block 112 may also include analyzing the structured data with the computer-readable artifacts to determine whether any relationships exist between them. In other embodiments, the analysis may simply append the structured data and the computer-readable artifacts for summarization purposes.

After the encounter bundle is analyzed in block 112, the analysis results are used to summarize the encounter bundle in block 114. In some embodiments, the summarization in block 114 may update a previously existing summary with the changes resulting from the patient data. In other embodiments, the summarization process may completely overwrite the previously existing summary with a new summary that reflects the patient data.

The specific summarization process in block 114 may vary depending on the embodiment, the use case, the data in the patient record, and other variables. For example, the summarization process may detect and identify or remove duplicate data in the encounter bundle; data that may be inaccurate, questionable, or obsolete; or data that may be out of date. As such, in some embodiments the summarization process may emphasize more recent data rather than older data. In some embodiments, data that is emphasized in the summary may be correlated with other unstructured and structured data in the encounter bundle. Sections identified in this correlation process (e.g., medication prescribed for an injury discussed at a recent doctor's visit) may then be emphasized in the summary as well.

In some embodiments, it may be beneficial for the summarization process to append information from external resources to the summary. For example, a computer system that managers an electronic medical record may have access to recent peer-reviewed medical journals, and may include the abstract of a recent journal article that presents findings regarding a disease that was discussed during the encounter with which the encounter bundle is associated. Further, the computer system may also review recent developments on prescribing medications for an affliction and place a note in the summary if a prescription written during that encounter does not comport with those recent developments.

In some instances, a medical practitioner may request a summary of the entire patient's medical record. In these instances, updating the summary may take the form of a merge operation that merges all the information available for the encounter bundles into one summary artifact. For example, the summary of the medical record may take the form of a directory or file (e.g., a compressed file) that contains the analysis results (e.g., results that are analogous to the results of block 112) from each encounter bundle. Thus, when the summary is updated in method 100, the results of block 112 may replace any previous results for that encounter bundle in the summary artifact. If, there are no prior analysis results for that encounter bundle (for example, if a new encounter bundle were created in block 110), the results of block 112 may be merged with the summary artifact in block 114. In this way, the results of prior analyses of encounter bundles that were not affected by the patient data may be reused.

In some embodiments, the summarization in block 114 may occur effectively instantly (e.g., as soon as the results of the analysis from block 112 are available), or may occur at a pre-determined time. For example, for medical records that are updated many times in a day (e.g., when a patient is seeing multiple doctors and having multiple tests performed in a day), it may be desirable to update the summary periodically (e.g., every 3 hours) rather than upon every update. Doing so may provide medical professionals with sufficiently real-time updates, but may enable the computer system to update the summary with changes to multiple encounter bundles at one time (i.e., after multiple iterations of blocks 102-112) which may save processing resources. In some embodiments, summaries may not be created (or updated) for an encounter bundle until a medical practitioner requests a summary for that particular encounter bundle. This may save processing power when new data is added to an encounter bundle that is not expected to be of any interest to a medical practitioner in the near future; the processing power that would otherwise be used to analyze that encounter bundle and update the corresponding summary may then be used for more pressing operations. Finally, in some embodiments, computer-readable artifacts created in block 112 may be deleted after the summary is updated in block 114. This may reduce the storage requirement of the medical record.

In some embodiments of the present disclosure, a computer system (such as the computer system capable of performing method 100) may contain a server that stores the information of a patient's medical record. In some such embodiments, this server may take the form of one or more databases or other distributed storage system.

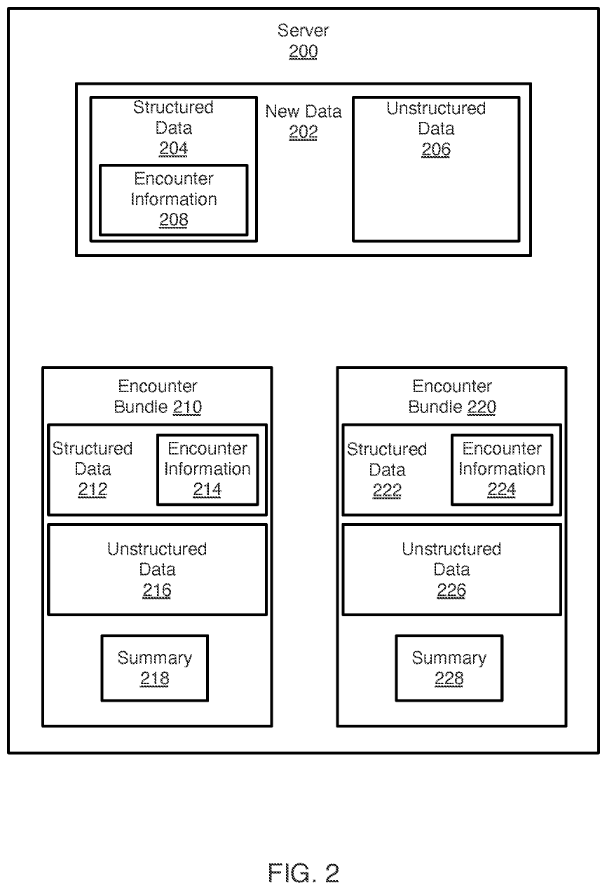

FIG. 2 illustrates a server 200 that may store the data of a patient's electronic medical record. When a computer system receives new data to be added to a patient's medical record, it may be received at server 200, or sent to server 200 for storage and processing. Thus, in addition to storing a patient's medical record, server 200 may also perform processing on the data therein, such as identifying encounters, creating encounter bundles, creating computer-readable artifacts from unstructured data, analyzing encounter bundles, and creating/updating summaries of the medical record.

Server 200 contains new data 202. New data 202 may be, for example, data that is received by a medical provider with regards to a recent patient visit. New data 202 contains structured data 204 and unstructured data 206. As illustrated, a portion of structured data 204 can be identified as encounter information 208. Encounter information 208, for example, may take the form of a day/time indication in a recognized field on new data 202. In many instances, encounter information may, as illustrated, be found in structured data. However, it is also possible for encounter information to be found in unstructured data. For example, a handwritten note made by a doctor during a medical exam may be signed and dated by the doctor, indicating the date of the medical exam and the name of the medical professional. From this information, an encounter may be derived.

Server 200 may also contain encounter bundle 210 and encounter bundle 220. Each of encounter bundle 210 and encounter bundle 220 may contain medical-record data associated with a particular bundle. In some embodiments, encounter bundle 210 and encounter bundle 220 may take the form of directories or compressed files. Encounter bundles 210 and 220 may also be labeled in a way that may quickly identify the encounter to which they correspond. Encounter bundles 210 and 220 also contain structured data 212 and 222. Structured data 212 and 222 may each contain encounter information 214 and 224. This encounter information may be information that was originally received as medical-record data, or may have been created by the server upon creating encounter bundles 210 and 220 (for example, encounter information 214 may take the form of a metadata tag created to label encounter bundle 210). Encounter bundles 210 and 220 also contain unstructured data 216 and 226. In some embodiments, unstructured data 216 and 226 may also contain information which may be used to identify an associated encounter. In some such embodiments, that information may be added to encounter information 214 and 224 as appropriate.

Upon receiving new data 202, a computer system (either part of server 200 or a computer system to which server 200 is connected) may attempt to identify a match between encounter information 208 and one of encounter information 214 and encounter information 224. If the computer system detects, for example, that encounter information 208 and encounter information 224 pertain to the same encounter, new data 202 may then be added to encounter bundle 220. In other words, encounter information 208 may be added to encounter information 224, structured data 204 may be added to structured data 222, and unstructured data 206 may be added to unstructured data 226.

As illustrated, encounter Bundles 210 and 220 also contain bundle summaries 218 and 228. In some embodiments, bundle summaries 218 and 228 may be summaries of the data within encounter bundle 210 and encounter bundle 220. In some embodiments, however, server 200 may not maintain summaries. Rather, the summaries of the encounter bundles may be located on a separate server, or may be created upon request and then destroyed when the resulting summary has been transmitted to the requesting medical professional. In some other embodiments, server 200 may also contain a summary of the entire medical record. In some embodiments, this summary of the entire medical record may be a merger of the analyzed data in encounter bundles 210 and encounter bundles 220, or a merger of summary 218 and summary 228.

As has been discussed, some embodiments of the present disclosure may utilize multiple different types of processing units in order to efficiently categorize and analyze medical-record data. For example, a generic central processing unit may be capable of efficiently identifying encounter information within newly received data and matching that encounter information with the encounter information in an encounter bundle. However, the same generic central processing unit may be ill fit for efficiently analyzing unstructured data and converting it to computer-readable artifacts. On the other hand, one or more graphics processor units may be capable of analyzing structured data very efficiently, but may be less efficient at categorizing medical data into encounter bundles.

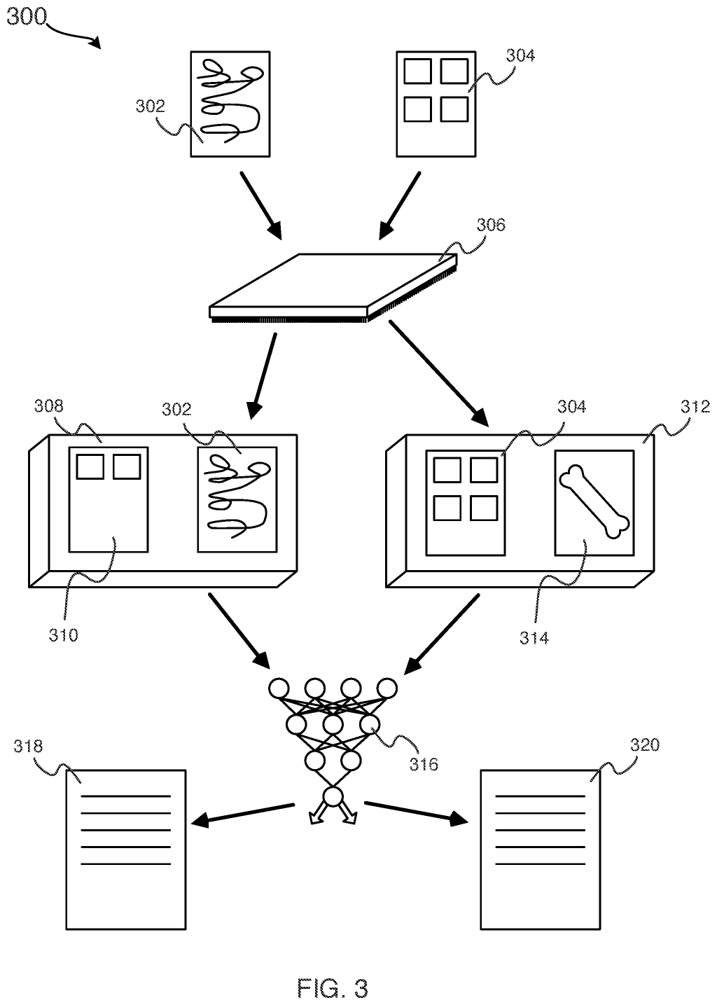

FIG. 3 illustrates an example flow diagram 300 in which two pieces of data are processed upon being added to a medical record. It is of note that flow diagram 300 is presented at a high level of abstraction for the sake of understanding; the sizes, proportions, and spatial relationships of the objects are not intended to be taken literally.

In flow diagram 300, handwritten note 302 and structured data 304 represent data that is being added to a medical record. In some embodiments, each of handwritten note 302 and structured data 304 may contain encounter information that could be used to categorize handwritten note 302 and structured data 304 into encounter bundles. For example, handwritten note 302 may include a metadata tag appended therein that may identify a doctor's visit associated with handwritten note 302.

Upon being received, handwritten note 302 and structured data 304 are processed by CPU 306. CPU 306 may be configured to identify encounter information in each of handwritten note 302 and structured data 304 and sort them into encounter bundles based on that encounter information. For example, as illustrated, CPU 306 determines, based on encounter information identified within handwritten note 302, that handwritten note 302 relates to the encounter associated with encounter bundle 308. Handwritten note 302 may then be added to encounter bundle 308, which also contains structured data 310. Similarly, CPU 306 determines, based on encounter information identified within structured data 304, that structured data 304 relates to the encounter associated with encounter bundle 312. Structured data 304 may then be added to encounter bundle 312, which also contains X-ray image 314. As illustrated, CPU 306 depicts a single CPU. In embodiments in which a single CPU determines the appropriate encounter bundle for new data, multiple cores or threads of that CPU may be used to do so. Further, where multiple instances of new data are added to the medical record simultaneously (or in close succession), each new instance of data may be assigned to a separate CPU core or thread.

As illustrated in FIG. 3, both encounter bundles 308 and 312 contain updated data. Thus, it may be beneficial to reprocess both encounter bundles 308 and 312 to update summaries of those encounter bundles. Thus, both encounter bundles 308 and 312 are processed by one or more AI analysis system 316 configured to perform data analysis on unstructured data. In FIG. 3, AI analysis system 316 is depicted by an abstracted illustration of a neural network, which may be running, for example, on one or more graphics processing units. However, in some embodiments, AI analysis system 316 may comprise other AI analysis systems, such as rule-based analysis systems.

Further, in some embodiments, both encounter bundles 308 and 312 may be processed by separate computer systems, separate paths of processing units within a graphics processing unit, or by separate graphics processing units entirely. Similarly, different pieces of data within each encounter bundle may also be analyzed using similarly separate hardware or software. For example, if encounter bundle 312 also contained the unstructured data of a second X-Ray image, the two X-ray images within bundle 312 may be analyzed by separate processing paths within a graphics processor.

AI analysis system 316 may analyze the unstructured data within encounter bundles 308 and 312 and convert it to computer-readable artifacts 318 and 320. In some instances, AI analysis system 316 may also analyze the structured data within encounter bundles 308 and 312 in order to properly digest and ascertain the meaning of the unstructured data.

In other words, computer-readable artifact 318 may comprise the information within handwritten note 302 converted into computer-readable form. In some embodiments, computer-readable artifact 318 may also comprise structured data 310. Similarly, computer-readable artifact 318 may comprise the information within X-ray image 314 converted into readable form, and may also contain structured data 304. In some embodiments, computer-readable artifacts 318 and 320 may be used to create a summary of each encounter bundle 308 and 312 (summary not illustrated in FIG. 3).

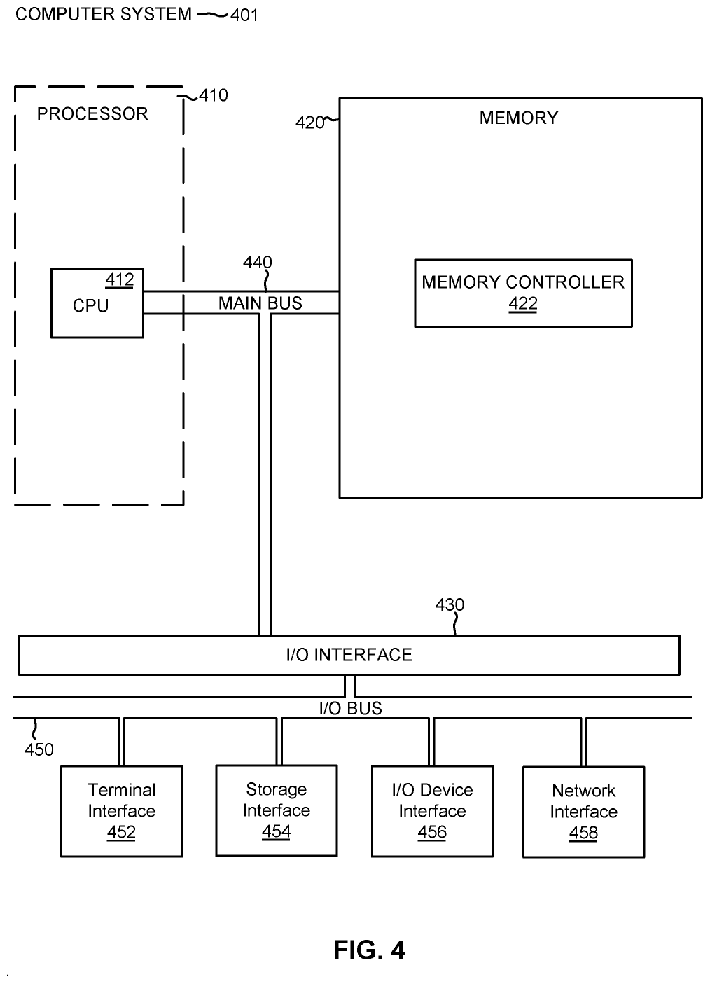

FIG. 4 depicts the representative major components of an example Computer System 401 that may be used in accordance with embodiments of the present disclosure. The particular components depicted are presented for the purpose of example only and are not necessarily the only such variations. The Computer System 401 may include a Processor 410, Memory 420, an Input/Output Interface (also referred to herein as I/O or I/O Interface) 430, and a Main Bus 440. The Main Bus 440 may provide communication pathways for the other components of the Computer System 401. In some embodiments, the Main Bus 440 may connect to other components such as a specialized digital signal processor (not depicted).

The Processor 410 of the Computer System 401 may include one or more CPUs 412. The Processor 410 may additionally include one or more memory buffers or caches (not depicted) that provide temporary storage of instructions and data for the CPU 412. The CPU 412 may perform instructions on input provided from the caches or from the Memory 420 and output the result to caches or the Memory 420. The CPU 412 may include one or more circuits configured to perform one or methods consistent with embodiments of the present disclosure. In some embodiments, the Computer System 401 may contain multiple Processors 410 typical of a relatively large system. In other embodiments, however, the Computer System 401 may be a single processor with a singular CPU 412.

The Memory 420 of the Computer System 401 may include a Memory Controller 422 and one or more memory modules for temporarily or permanently storing data (not depicted). In some embodiments, the Memory 420 may include a random-access semiconductor memory, storage device, or storage medium (either volatile or non-volatile) for storing data and programs. The Memory Controller 422 may communicate with the Processor 410, facilitating storage and retrieval of information in the memory modules. The Memory Controller 422 may communicate with the I/O Interface 430, facilitating storage and retrieval of input or output in the memory modules. In some embodiments, the memory modules may be dual in-line memory modules.

The I/O Interface 430 may include an I/O Bus 450, a Terminal Interface 452, a Storage Interface 454, an I/O Device Interface 456, and a Network Interface 458. The I/O Interface 430 may connect the Main Bus 440 to the I/O Bus 450. The I/O Interface 430 may direct instructions and data from the Processor 410 and Memory 420 to the various interfaces of the I/O Bus 450. The I/O Interface 430 may also direct instructions and data from the various interfaces of the I/O Bus 450 to the Processor 410 and Memory 420. The various interfaces may include the Terminal Interface 452, the Storage Interface 454, the I/O Device Interface 456, and the Network Interface 458. In some embodiments, the various interfaces may include a subset of the aforementioned interfaces (e.g., an embedded computer system in an industrial application may not include the Terminal Interface 452 and the Storage Interface 454).

Logic modules throughout the Computer System 401—including but not limited to the Memory 420, the Processor 410, and the I/O Interface 430—may communicate failures and changes to one or more components to a hypervisor or operating system (not depicted). The hypervisor or the operating system may allocate the various resources available in the Computer System 401 and track the location of data in Memory 420 and of processes assigned to various CPUs 412. In embodiments that combine or rearrange elements, aspects of the logic modules' capabilities may be combined or redistributed. These variations would be apparent to one skilled in the art.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to explain the principles of the embodiments, the practical application or technical improvement over technologies found in the marketplace, or to enable others of ordinary skill in the art to understand the embodiments disclosed herein.

What is claimed is:

1. A method comprising:
receiving, by a computer system that manages an electronic medical record, patient data associated with the medical record;
identifying first encounter information in the patient data;
comparing the first encounter information with second encounter information in a first encounter bundle in the medical record;
determining, based on the comparing, that the first encounter information and the second encounter information are associated with the same encounter;
adding, based on the determining, the patient data to the first encounter bundle;
analyzing the first encounter bundle, resulting in a computer-readable artifact for the first encounter bundle; and
updating, based in part on the computer-readable artifact, a summary of the first encounter bundle.

2. The method of claim 1, further comprising discarding the computer-readable artifact after the updating.

3. The method of claim 1, wherein a summary of a second encounter bundle is unchanged by the updating.

4. The method of claim 1, wherein the adding further comprises updating a version number of the first encounter bundle.

5. The method of claim 1, wherein the analyzing and updating occur at a pre-determined time.

6. The method of claim 1, wherein the analyzing and the updating occur when a summary of the first encounter bundle is requested by a medical practitioner.

7. A system comprising:
a processor; and
a memory in communication with the processor, the memory containing program instructions that, when executed by the processor, are configured to cause the processor to perform a method, the method comprising:
receiving first patient data associated with a medical record;
identifying first encounter information in the first patient data;
comparing the first encounter information with second encounter information in a first encounter bundle in the medical record;
determining, based on the comparing, that the first encounter information and the second encounter information are not associated with the same encounter;
creating, based on the determining, a second encounter bundle in the medical record;
adding, based on the determining, the first patient data to the second encounter bundle;
analyzing the second encounter bundle, resulting in a first computer-readable artifact for the second encounter bundle; and
creating, based in part on the first computer-readable artifact, a summary of the second encounter bundle.

8. The system of claim 7, further comprising:
receiving second patient data associated with the medical record;
identifying third encounter information in the second patient data;
comparing the third encounter information with the first encounter information and the second encounter information;
determining, based on the comparing, that the third encounter information and the first encounter information are is associated with the same encounter;
adding, based on the determining, the second patient data to the second encounter bundle;
analyzing the second encounter bundle, resulting in a second computer-readable artifact for the second encounter bundle; and
updating, based at least on the second computer-readable artifact, summary of the second encounter bundle.

9. The system of claim 8, wherein the adding further comprises updating a version number of the second encounter bundle.

10. The system of claim 7, further comprising discarding the computer-readable artifact after the updating.

11. The system of claim 7, wherein the summary of a third encounter bundle is unchanged by the updating.

12. The system of claim 7, wherein the analyzing and the creating occur when a summary of the first encounter bundle is requested by a medical practitioner.

13. The system of claim 7, wherein the analyzing and creating occur at a pre-determined time.

14. A computer program product, the computer program product comprising a computer readable storage medium having program instructions embodied therewith, the program instructions executable by a computer to cause the computer to:
receive first patient data associated with a medical record;
identify first encounter information in the first patient data;
compare the first encounter information with second encounter information in a first encounter bundle in the medical record;
determine, based on the comparing, that the first encounter information and the second encounter information are not associated with the same encounter;
create, based on the determining, a second encounter bundle in the medical record;
add, based on the determining, the first patient data to the second encounter bundle;
analyze the second encounter bundle, resulting in a first computer-readable artifact for the second encounter bundle; and
create, based in part on the first computer-readable artifact, a summary of the second encounter bundle.

15. The computer program product of claim 14, wherein the program instructions further cause the computer to:
receive second patient data associated with the medical record;
identify third encounter information in the second patient data;
compare the third encounter information with the first encounter information and the second encounter information;
determine, based on the comparing, that the third encounter information and the first encounter information are is associated with the same encounter;
add, based on the determining, the second patient data to the second encounter bundle;
analyze the second encounter bundle, resulting in a second computer-readable artifact for the second encounter bundle; and
update, based at least on the second computer-readable artifact, summary of the second encounter bundle.

16. The computer program product of claim 15, wherein the adding further comprises updating a version number of the second encounter bundle.

17. The computer program product of claim 15, wherein the program instructions further cause the computer to discard the computer-readable artifact after the updating.

18. The computer program product of claim 15, wherein the summary of a third encounter bundle is unchanged by the updating.

19. The computer program product of claim 15, wherein the analyzing and the creating occur when a summary of the first encounter bundle is requested by a medical practitioner.

20. The computer program product of claim 15, wherein the analyzing and creating occur at a pre-determined time.
